(19) United States
(12) Patent Application Publication
(10) Pub. No.: US 2014/0311218 A1
(43) Pub. Date: Oct. 23, 2014

(54) Methods of Producing Pseudoscent Compositions of Narcotic Materials and Compositions Thereof

(71) Applicant: David Adebimpe, Annapolis, MD (US)

(72) Inventor: David Adebimpe, Annapolis, MD (US)

(21) Appl. No.: 14/217,431

(22) Filed: Mar. 17, 2014

Related U.S. Application Data
(60) Provisional application No. 61/802,312, filed on Mar. 15, 2013.

(51) Int. Cl. G01N 33/00

(52) U.S. Cl. CPC G01N 33/0001; G01N 2033/0003
USPC 73/23.34; 252/408.1

(57) ABSTRACT

This invention relates to a method that can be used to scientifically fabricate pseudoscents of narcotics, which, in their entirety, are comprised of non-narcotic materials. It also discloses specific compositions of such pseudoscents, which can be used as narcotic-free-but-odoriferously-identical simulants for a range of narcotics and are comprised of non-narcotic components of the scent signature of a narcotic and/or scent components of the same headspace scent signature that have narcotic qualities but have been rendered non-narcotic. The scents achievable by the groups of formulations generated by this method include the different types of prohibited and controlled narcotics, and the components within the pseudoscents can be further tuned to generate simulants representative of different qualities and quantities of such narcotics. These non-narcotic scent simulants can be used to bolster existing narcotic detector-dog training programs, establish new training paradigms in canine, rodent, insect, and other creature narcotic detection and training and, in some cases, increase the efficiencies of analytical instruments that rely on the phenomenon of vapor sampling to detect narcotic materials.

### CROSS REFERENCE TO RELATED APPLICATIONS

This application claims priority from U.S. Provisional Application 61/802,312, titled a System and Methods of Producing Pseudoscent Compositions of Narcotic Materials and Compositions Thereof, filed on Mar. 15, 2013, the entirety of which is hereby incorporated by reference.

### STATEMENT REGARDING FEDERALLY SPONSORED RESEARCH

Not Applicable

### BACKGROUND OF THE INVENTION

The present invention relates to a method for producing non-narcotic scent simulants of narcotics (otherwise known as “pseudoscents”) that smell like real narcotics but lack any narcotic material and lack and of the physiological or psychedelic characteristics of real narcotics. These non-narcotic scent simulants can be used to compliment or supplant the use of real narcotics in the training and evaluation regimen of narcotic-detecting dogs and other creatures. It also relates to a method for validating such pseudoscents, and defines the compositions of some pseudoscents simulants.

Currently, Non-narcotic scent simulants or pseudoscents of narcotics are based upon scientific principles that involve increasing the surface area of such narcotics in attempt to increase the dispersivity of the smell. This is achieved by using highly dispersed cohorts of the real narcotics or narcotic particulates rather than using the principles based on investigative enquires into the underpinnings of the science of odors and their relationship with the olfactory capacity of narcotic-detection creatures.

Accordingly, there is a continuing interest in the development of narcotic-scent simulants that do not contain any of the physiological or psychedelic properties of real narcotics. This invention can be used to manufacture non-narcotic scent simulants of narcotics that do not contain all the psychedelic properties of the narcotics the real narcotics and confer several advantages over real narcotics: non-narcotic scent simulants of narcotics can be safely transported without the attendant risks of transporting real narcotics; non-narcotic scent simulants can be handled without the worry of getting a permit from a regulatory agency; and, from a logistical standpoint, drug detector programs can easily obtain non-narcotic scent simulants without the hassle of obtaining real narcotics.

### BRIEF SUMMARY OF THE INVENTION

The present invention relates to a method that can be used to produce non-narcotic scent-simulants of narcotics that smell so similar to the narcotic being simulated that both scents are indistinguishable from each other to a narcotic-detecting creature. Such compositions of pseudoscents can be used for training and evaluating creatures, such as dogs, in narcotics detection. The present invention also further relates to compositions of pseudoscents that have no narcotic components whatsoever but whose scents nonetheless simulate the scents of narcotics such as cocaine, heroin, methamphetamine, PCP, synthetic cannabinoids, cannabis, opium, LSD, and MDMA. These scents have no direct physical or chemical equivalence to the narcotics that they simulate; they are solely of odoriferous equivalence.

Also, as none of the pseudoscent components can be classified as narcotic material, these pseudoscents can also be stored, transported and deployed using methods and forms, which, as narcotics, would be hazardous, would require a license, would be illegal, or would be entirely impossible. The pseudoscents produced by this method are suitable for the training of canines, as well as other scent-detecting creatures, to detect narcotics and narcotic-containing materials and products, with each pseudoscent type containing carefully selected odoriferous markers that define a particular narcotic. Using these pseudoscents in conjunction with an effective narcotics-detection training regime will allow narcotics-detecting creatures such as dogs, bees, rats and fishes to achieve detection efficiencies that are far superior to those achieved by training on both real narcotics and other scent-simulants containing real narcotics. This is because the principal components needed for detection have been scientifically identified, isolated from the headspace scent signature of each narcotic and odoriferous equivalents have also been determined. The identified and determined components have, thereafter, been formulated into a pseudoscent whose scent components are known and can be precisely controlled.

A method for making a non-narcotic scent simulant that smells like the simulated real narcotic but does not have the narcotic characteristics of the simulated narcotic comprising: a) targeting a narcotic material for detection; b) identifying the odor components within the headspace scent signature of the narcotic that is targeted for detection; c) identifying components that are odoriferously similar to the headspace components within the headspace scent signature of the material that is targeted for detection; d) combining components identified in b) into a first formulation; e) combining the components identified in c) into a second formulation; and f) combining the components identified in b) and c) into a third formulation, wherein the non-narcotic pseudoscent is substantially free of the narcotic.

In one embodiment, the structural framework of a real narcotic is substituted with a larger or a differently-configured one,

In another embodiment, the functional group of real narcotic is substituted with a functional group that has similar electronic properties, thereby diluting the pharmacologically-active potential of the narcotic component of the headspace scent profile to the extent that it loses its narcotic character.

In another related embodiment, the structural backbone of the narcotic molecule is supplanted with a homologue that has a higher molecular weight in an attempt to nullify or dilute the narcotic effects of the functional group.

In yet another embodiment, the functional group(s) within the molecular structure of the headspace component is replaced with a non-pharmacological active functional group(s) that has similar electron donating or withdrawing properties.

In another embodiment, the ratios of odoriferous components within the non-narcotic scent simulant is adjusted to duplicate the scent of varying amounts of the narcotic.

### DEFINITION OF TERMS

A non-narcotic scent simulant of a narcotic is a substance that produces a scent that is so similar to the scent of the narcotic it is supposed to simulate that neither scent can be differentiated from one another by a narcotic detecting creature. Such a non-narcotic scent simulant of a narcotic can also be referred to as the odoriferous equivalent of that narcotic.

A non-narcotic pseudoscent is a substance that comprises of one or more scent simulant components. However, all the materials used in its manufacture, including the scent it emanates, are not comprised of the narcotic being simulated.

### DETAILED DESCRIPTION OF THE INVENTION

The present invention relates to pseudoscents of narcotic materials, which are essentially scent simulants of a narcotic that does not contain any narcotic materials themselves. These pseudoscents will be particularly useful in the evaluation and training and of narcotics detecting creatures such as canines, bees and rats.

Known for their acute sense of smell, canines have been used to perform various forms of scent-based detection work: the search and rescue of missing, injured or deceased persons; the detection of narcotics and drugs by police and federal law enforcement authorities; the detection of accelerants in arson investigation; the detection of moulds and other biohazards; and in the detection of explosives, firearms, ammunition and mines. When fully trained, a typical canine can search a car per minute and over 400 packages in half an hour. Furthermore, unlike the point-detection capability of narcotic-detection instruments and machines, canines can pick up a scent and track it to its source. In a search-and-detect situation, a trained narcotics-detection canine will “key” (i.e., identify and/or detect) onto specific scents, which, to a human, are seemingly indistinguishable from other scents present in the environment, and trace the scent to the material that is producing such odor. However, even with such superb discriminatory capacity, such dogs miss a small-but-significant percentage of target material during their search process. These failures are caused by a number of factors, the most pertinent being improper foundation training, the use of wrong aids in detector-dog training programs, and the lack of proper training aids. In order to decrease these failure rates, new and technically superior scent simulants are needed.

In these modern times, some trainers believe that real narcotics are better than non-narcotic scent simulants and prefer to use real narcotics as training aids over non-narcotic scent simulants. This preferential use is based on their belief that it is impossible to duplicate the odor of a narcotic to a level of accuracy sufficient for scent-detection raining without using a narcotic component. Thus, since cocaine will contain only cocaine within its odor profile and marijuana odor essentially contains cannabinoids, non-narcotic scent simulants will not have the efficacy of using the “real thing” as the training aid for its own detection. From this simple premise, which translates to “what we see is what we smell,” deductions were advanced that canines must therefore be trained on the scent of only the material that it is required to detect in order to successfully imprint the canine with the narcotic it is trained to detect. What is overlooked is the fact that for humans, other mammals, and a host of other creatures, what is seen is not necessarily what is smelt. In the first instance, as different creatures see same object to different resolutions; they smell the same object to different resolutions, too. However, and more importantly, whatever it is they smell is not simply the material that is being seen, but the components within the material that is (are) volatile enough to have the affinity to exude from the material being seen. Moreover, the concentration of each volatile material within the scent signature of the material must be equal to or greater than the creature's olfactory threshold, which is the lowest olfactory stimulus intensity a creature can detect before such volatile component can be picked up by the sense of smell as being present within the headspace. Due to differences in physiology, anatomy, and affinities of olfactory organs, and in the number and densities of olfactory cells, it is also expected that such olfactory threshold will itself, differ from creature to creature. This makes it possible for different creatures to identify the same material using different components of the scent signature of volatiles exuded by a material, and not the total number of components within the scent signature itself.

Our findings show that, in most instances, non-narcotic components of a narcotic normally resulting from the decomposition of such narcotics are more volatile than the narcotic itself. The highly volatile nature of some of these non-narcotic components means that they will be omnipresent within the scent signature of narcotics and this makes them better odoriferous markers for a narcotics detection program based on olfaction than using the narcotic itself as a detection marker, as they will be easier to detect by olfactory receptors involved in the sense of smell. The function of the nose is based on vapor sampling which corresponds to single-molecule sampling and not, as thought, particulate sampling. Furthermore, engaging these non-narcotic scents as training aids within the training regimen of, for example, narcotics detecting canines, will greatly improve their success rate in detecting narcotics.

Using marijuana as an example, it is believed by the scent-detection community that its pharmacologically-active ingredients, terahydrocannabinol (known as THC), cannabidiol, and tetrahydrocannabivarin are the main odor markers that scent detecting canines use in its detection. However, this supposition stems from the fact that these are the ingredients that make marijuana illegal. In actuality, the low vapor pressure of THC and these other pharmacologically-active constituents cannot significantly contribute to the headspace scent signatures of marijuana mainly because of their high molecular weights and, therefore, high vapor pressures. Moreover, when combined with other materials such as binders and starches, as in the case of methylenedioxymethamphetamine (MDMA), which is normally used as a training aid in its tablet form, the scent signature of the narcotic being sought is being contaminated with the materials used to make the narcotic into a tableted form. These materials change from manufacturer toi manufacturer and so does the scent signature—even if the narcotic component within the tablet is of the same type and quantity.

Non-narcotic scent simulants and pseudoscents that are fabricated for research and developmental work in narcotics detection will be more effective if their formulations embrace both the de facto constituents of the headspace scent of the narcotic material and addresses the olfactory competence of the narcotics detecting canine, rather than solely concentrating on finding the narcotic component within a narcotic. A narcotic is for the sense of sight while its odor is for the sense of smell. Such considerations will result in the formulation of simulants that can actually be used to develop, sharpen, and/or evaluate the abilities of narcotics-detecting creatures and, when used to develop logic algorithms, increase the detection rate of analytical instruments.

The present invention therefore relates to a method suitable for fabricating pseudoscents of narcotic materials which, albeit precluding any narcotic material, contain the necessary odoriferous markers that characterizes a particular narcotic. This method involves the process of reconstituting the experimentally determined headspace scent signature of a narcotic into a formulation that retains all non-narcotic components within such a signature and substitutes the narcotic components, if any, with equivalents whose pharmacologically-active group(s) has been substituted with a “non-narcotic but odoriferously equivalent” group(s).

The foundation of this invention hinges on the supposition that the physicochemical composition of an narcotic material is of minor significance in the fabrication of an narcotic simulant for the training and evaluation of narcotic-detecting creature and in the development of narcotics-detecting analytical instruments; what is more important is its scent—which can be determined by the initial (analytical) identification of the headspace scent signature of these narcotic materials—as this is essentially what the diversity of narcotic detecting creatures such as dogs, rodents and bees detect.

The present invention also relates to compositions of pseudoscents for scent simulants that contain no narcotic components whatsoever but are identical to the scents of materials that are designated as narcotics by the US Drug Enforcement Administration (DEA). Such materials include cocaine, heroin, methamphetamine, marijuana, phencyclidine (PCP), and amphetamine-based narcotics. These pseudoscents have no direct physical or chemical equivalence to the narcotic materials or compositions they simulate; they are solely of odoriferous equivalence. Also, since they are non-narcotic, none of the pseudoscent formulations produced by the present method can be classified as a narcotic material and the pseudoscents can be stored, transported, and implemented as a training aid, using methods and forms, which, as real narcotics, would be hazardous, require a license, or impossible. The pseudoscents produced by this method are also suitable for the training of search-and-detect creatures that use the element of scent-detection for their detection activities and, when applicable, the calibration of analytical instrumentation that relies on the principle of vapor sampling to detect narcotic materials. Using these compositions with an appropriate training regime will allow narcotic detecting creatures, such as dogs, to achieve efficiencies that are far superior to analytical instruments or to if they were trained using real narcotics. By adjusting the types and ratios of odoriferous components within the pseudoscent, it is also possible to further tune the pseudoscent to duplicate the scent of varying amounts of the narcotic it simulates. The compositions may also be used to train other creatures, for example, bees, rodents and wasps.

TABLE 1
Chemical structures of some Narcotics

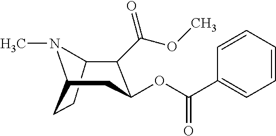

Cocaine

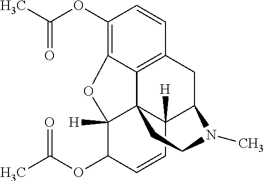

Heroin

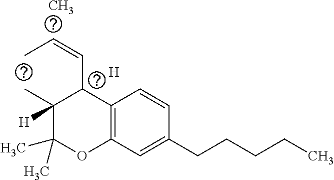

Marijuana (Tetrahydrocannabinol,
active ingredient shown)

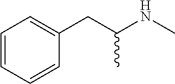

Methamphetamine

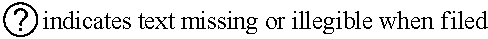

TABLE 2
An illustration of the interrelationships between a simulated narcotic,
its non-narcotic pseudoscent formulation and the scope of detection
of the pseudoscent, if used as a narcotic-detection training aid.

| | | | Example of Scope of |
|---|---|---|---|
| | | | narcotic detection |
| | | | using specific |
| Specific explosive | | Examples of specific | pseudoscent |
| component being | Examples of Identified | pseudoscent | formulation as a |
| simulated | Pseudoscents | formulations | training aid |
| Cocaine | Methyl cinnamate, | 2 g methyl cinnamate + | Cocaine, crack cocaine |
| | cinnamic acid, | 2 g benzoic acid + | |
| | benzoic acid | 10 g diatomaceous earth | |
| Heroin | Acetic acid, phenyl | 4 g phenyl acetate + | Heroin, opium and |
| | acetate, acetophenone | 10 g diatomaceous earth | opium soaked tobacco |
| Methamphetamine | Benzaldehyde, | 1.5 g benzaldehyde + | All forms of amphetamines, |
| | benzylmethylamine, | 2 g propiophenone + | “crystal meth” |
| | propiophenone | 10 g cellulose | |
| PCP | Phenylcyclohexene, | 4.40 g phenylcyclohexene + | PCP |
| | cyclohexlpiperidine, | 2 g + piperidine + | |
| | piperidine | 15 g diatomaceous earth | |
| Marijuana | β-caryophellene + | 2.20 g β-caryophellene + | Marijuana, “spice”, |
| | myrcene | 3.65 myrcene + | “K2” |
| | | 20 g cellulose | |
| MDMA | Phenyl acetic acid, | 2 g phenylacetic acid + | MDMA, in pure |
| | benzodioxole, | 2 g benzodioxole | and tablet forms |

A method of forming an odoriferously-identical and non-narcotic analogue of a component found within the headspace scent signature of a narcotic while retaining the odoriferous characteristics of such a component is attained through a substitution of the structural framework of such headspace component with a higher homologue, or homologues, which harness electronic properties similar to the parent molecule of such component. Through such substitution of the structural framework with a larger or a differently-configured one, or through the substitution of a functional group with one which has similar electronic properties, the narcotic and pharmacologically-active potential of a narcotic component of the headspace scent profile of a narcotic can be diluted to the extent that it loses its narcotic character, since the ratio of the pharmacologically-active functional groups to the whole molecule, or its ability to coordinate into an narcotic entity, decreases. For example, methamphetamine can be made to lose its narcotic capacity,] but retain its odoriferous quality by replacing the cyclic-and-aromatic benzene structural backbone with the linear-and-conjugated hexatriene moiety.

Thus, in one embodiment, the non-narcotic pseudoscent can include non-narcotic components identified within the headspace scent signature of the narcotic, as a distinct formulation. Suitable examples of non-narcotic components of the analyzed headspace scent signature of narcotics that are suitable for formulation into a non-narcotic scent simulant, include benzaldehyde for methamphetamine, pinene for marijuana, and cyclohexlpiperidine for PCP.

In another embodiment, the non-narcotic pseudoscent can include odoriferously identical equivalents of the headspace components, as a distinct formulation. These equivalents are essentially analogues of components of the headspace that have undergone structural modification at a molecular level through either (A) a successive removal of their pharmacologically-active functional groups until the molecule is rendered non-narcotic in character, or (B) supplanting the structural backbone of the narcotic molecule with a homologue that has a higher molecular weight, in an attempt to dilute or nullify the narcotic effects of the functional groups, and/or by (C) replacing the functional group(s) within the molecular structure of headspace component with a non-pharmacologically active functional group(s) that has similar electron donating or withdrawing properties. It is disclosed that these techniques will essentially render the molecule non-narcotic while retaining the odor characteristics of the parent molecule.

In another embodiment the non-narcotic scent simulant pseudoscent can include combinations of both headspace and odoriferously-identical components of the headspace scent signature of a narcotic as a distinct composite formulation.

The formation of the pseudoscent is itself achieved by simply dispersing, at low concentration, amounts of the headspace and/or odoriferously-identical components(s) of the scent signature of a narcotic material, as deduced from headspace analysis of the material, within an inert matrix. As illustrated in Table 2, it is not necessary that all the determined non-narcotic or odoriferously-identical components of the headspace scent signature be used within a first, second or composite formulation. Preferably any of the components within a headspace scent signature that has a concentration that falls within the minimum and maximum olfactory thresholds of the narcotic detecting creature can be used within the formulation. Those components with lower vapor pressures can be particularly useful in the formulation of pseudoscents that will aid in the detection of equivalents of large amounts of narcotics. This is because, in large amounts of narcotics, the emanation of components of higher vapor pressures is so rapid that they can completely saturate a room, thereby eliminating the availability of an odor concentration gradient and resulting in the inability of the canine to trace the odor to its source. Being exposed to an area saturated with the odor for prolonged periods may also result in (a reversible) desensitization towards such odor components during a search process, resulting in a difficulty in locating the target narcotic. So, the low vapor pressure components within the narcotic scent signature will be more appropriate to be used as a training aid in this instance. Conversely, components within the headspace signature with higher vapor pressure can be particularly used in the formulation of pseudoscents that will aid in the detection of small amounts of narcotics. This is due to the fact that, when small amounts of narcotics are used, the headspace scent signature is dominated by the components with high vapor pressure, and these will be suitable as target scents within a training program.

The matrix used for the pseudoscent formulation may be solid, liquid or gaseous. An example of a gaseous matrix is an aerosol. Another example is a non-reactive porous support that allows for a controlled or slow release of the components within the pseudoscents. Depending on the characteristics of the narcotic scent simulant, cross-linked synthetic polymer (e.g. silica, cellulose), gels, emulsions, hydrogels, fillers (diatomaceous earth, clay, grain husks, saw-dust, porous beads, grain husks, natural fibers), bio-organic polymers, for example, may be used a dispersant matrices. The microstructure of the matrices may be such that it is amorphous or defined. Examples of matrix morphologies include spray dried power, a sphere (e.g. balls, pebbles, microspheres or a pellet). The matrix can also include other polymers, buffers, salts, or fillers. The pseudoscent formulants may also be adsorbed onto an inert matrix that has intestacies or pores with diameters that are greater than the longitudinal cross-section of the smallest odoriferous substance within the formulation. Such a matrix enables adsorption of the narcotic scent simulant within the pores rather than the surface, and their slow release from the confines of the interstices, rather than their evaporation off the absorbent, thus decreasing any chances of accelerated decomposition due to surface-area catalysis.

The pseudoscent formulation can include a binder. Such a binder can include a polymer or a compound that has a molecular weight of ≧320 atomic mass units. The preferable solvent to aid in the binding can be selected through using the following hierarchy: the lowest boiling liquid within the formulation as derived from the constituents of the headspace, water, or an organic solvent with a boiling point ≦75° C. at normal atmospheric pressure. The pseudoscent components, solvent, and the polymer are blended together and the solvent is thereafter evaporated under vacuum.

Such methods can be applied towards the formulation of pseudoscents for a variety of narcotics. Suitable narcotics can be cocaine, heroin, methamphetamine, phencyclidine, marijuana (cannabis), and methylenedioxymethamphetamine (MDMA).

Compositions

Pseudoscent compositions comprising: a plurality of non-narcotic components of the headspace scent signature of narcotics, and/or “odoriferously identical” equivalents of the components of the headspace scent signature of narcotics, the composition being free of narcotics.

Method of Evaluating Candidate Pseudoscents

A method of evaluating the efficacy of formulated pseudoscents, also referred to as a scent validation process, is advanced which comprises exposing a candidate pseudoscent to a creature that has already been trained in narcotic detection using real narcotics as a training aid, such as a certified narcotics-detecting dog. The response of the creature will determine if it can distinguish the scent of the pseudo from the scent of a real narcotic, and this in turn will determine if the candidate pseudoscent is suitable as a narcotic training aid for such a creature. A creature responds by displaying behavioral cues that indicate to the handler that the creature recognizes the scent it has been trained to recognize. For example, dogs respond by sitting down next to the scent it was imprinted to find. An ability to distinguish between both scents will mean that the pseudoscent has a scent that is different from the narcotic, which will deem it unsuitable for use as a narcotic training aid. If the response given is not different from that which the creature gives in response to the presence of a real narcotic material, then the pseudoscent can be deemed suitable for use as a training aid. This method of evaluation is used in all examples 3 to 10, where dogs were used to evaluate the suitability of non-narcotic components and odoriferously-similar derivatives of the headspace scent signature of narcotics as simulating the scent of actual narcotics.

Method for Producing a Non-Narcotic Pseudoscent of a Narcotic

A method for producing a non-narcotic scent simulant for narcotics is broadly contemplated. A method for producing an non-narcotic scent simulant of a narcotic comprising: a) targeting a narcotic material for detection; b) identifying the odor components within the headspace scent signature of the narcotic that is targeted for detection; c) identifying components that are odoriferously similar to the headspace components within the headspace scent signature of the material that is targeted for detection; d) combining components identified in b) into a first formulation; e) combining the components identified in c) into a second formulation; and f) combining the components identified in b) and c) into a third formulation, wherein the non-narcotic scent simulant is free of the narcotic.

The invention disclosed herein is exemplified by the following preparations and examples, which should not be construed to limit the scope of the disclosure. Alternative preparations and analogous structures may be apparent to those skilled in the art.

### Example 1

### Method of Identifying the Potential Components of a Narcotic Pseudoscent, and Subsequent Pseudoscent Formulation

The first stage of identifying the potential candidates for a non-narcotic scent simulant is to subject the narcotic to an analytical method that identifies the components of the headspace scent signature of the narcotic. A typical procedure of identifying this headspace signature is through the use of gas chromatography (GC) coupled to a mass spectrometer (MS). To achieve this, the narcotic of interest is placed in a sterile flask fitted with a serum cap equipped with a Solid Phase Micro Extraction (SPME) fiber that protrudes into the flask. Time is allowed for its scent to occupy the headspace, and the scent that has occupied the headspace equilibrates with the air inside the flask, and it is absorbed by the SPME fiber. The fiber is thereafter removed from the flask and placed into the inlet of the gas chromatography machine, heated, and desorbed. The desorbed scent travels into the GC column where its components are separated, and each of the separated components of the scent are identified. This identification process is based on the different retention times of the scent components due to their chemical nature, and it is typically an automated search against a compiled database of compounds with analyzed retention times. Identified components are further confirmed by the mass spectrometer part of the instrument, which further identifies the components based on their molecular mass. It is thereafter compared against a database of compounds of known mass and fragmentation patterns. Once the individual components of the scent signatures are known, the non-narcotic components can be directly used to formulate a pseudoscent, after the scent validation process using dogs that have been officially certified in the process of narcotics detection. Typically, those component scents identified within the headspace with vapor pressures that are sufficiently high enough to allow the manufacture of a formulation that provides headspace concentrations that are above the olfactory threshold limits of a dog, or any other creature used for detection, can be used. Pseudoscents can be formulated to produce a material with a scent profile of components in the same ratio as the original components are within the headspace of the real narcotic. Such ratios can be determined using GC methods. Pseudoscent formulation is achieved by simply dispersing selected non-narcotic components of the headspace and of odoriferously-identical equivalents of both non-narcotic and narcotic components, within an inert matrix, separately, or as a composite formulation, and in concentrations that will not allow the odor of the pseudoscent to be non-effective or overwhelming during its use.

### Example 2

### Method of Validating (Evaluating) a Narcotic Pseudoscent, after its Formulation

For testing the suitability of components as a potentially useful narcotic pseudoscent, a canine search-and-detect methodology has been developed that embraces the best practices in scent detection. The setup typically comprises of a 7×9 (63-position), or 6×6 (36-position) grid of cardboard or wooden boxes, of 1 ft³ in volume and spaced at least 4 ft apart, each of which has a 5-inch diameter opening cut out of the topside to allow easy sniffing of the box by canines. Other dimensions or box arrays or box arrangements can be used, depending on the number of candidate odors available for testing. Non-narcotic components and their pseudoscent equivalents, including the pseudoscent equivalents of the narcotic components, as determined from headspace analysis of the narcotic, were dispersed within the inert matrices, put in jars, and then randomly placed into each box, through the opening. Also randomly placed within the test grid were real narcotics such as cocaine, heroin, and MDMA. These are to be used to determine the dog's ability to detect real narcotics under the same conditions as the candidate scents. Distracters such as food and toys were also planted in some boxes within the grid to help ascertain the propensity of the dogs in disregarding these objects. Each test session was configured in a way that within each test session, there were more non-targets (empty boxes) than targets (occupied boxes), at least an empty box between two targets, and no more than eight candidate pseudoscents were placed within a test grid per trial session. After the samples are placed and their placement noted, a period of at least 5 minutes was allowed for the vapors of the sample to diffuse in to the box. The maximum and minimum concentration of headspace vapor that can be achieved within a box are controlled through the formulation process, which takes into consideration the vapor pressure and mass ratio of the formulation component(s) to their matrix, the prevailing temperatures, and the scent generation rate. The longevity of the scent can also be determined if the evaporation rate(s) of the formulation component(s) the type and amount of dispersing matrix used, and the surface area of the container holding the formulation are known. After the standing period, the certified dog/handler teams were successively allowed into the grid to commence a search for narcotics hidden within the grid. At least four dog/handler teams were used during each test. All dogs used in the test process were certified, experienced narcotic detecting dogs and none of the dog/handler teams knew the boxes that contained the candidate scents, the real narcotics, or the distracters. For a true double-blind test, even the test-supervisors will be unaware of the location of these items, either. As a team enters the grid, the handler systematically guides the canines around the grid while the canine sniffs around the perimeter and through the opening on the top side of each box, seeking for narcotics that it has been trained to find. This process validates a candidate scent as being a non-narcotic scent simulant. A candidate scent is positively validated as a potential non-narcotic-scent simulant when the previously-certified dog assuredly and consistently sits next to the box within which it is contained, as this means that the dog supposes that the scent emanating from the box is that of a real narcotic. Such assuredness, if consistently displayed by experienced narcotics detecting dogs, means that the material within the box being validated has a scent that is so similar to that of a narcotic that it cannot be differentiated from a real narcotic by a dog certified in the art of narcotics detection. When a candidate pseudoscent is positively validated, it can then be used within a narcotics-detection program as a general training aid to train the narcotics-detection dog instead of using the real narcotic it represents. It can also be used as a specialty training aid to hone narcotic-detector dogs onto more precise components of an odor during narcotics detection. To date, the true scent signatures of typical narcotics are unknown and neither are the components of a narcotic scent that optimizes the efficacy of narcotic-detector dogs in its detection process.

After the end of a test run when all dog/handler teams have each undergone a complete run of the grid, the dog/handler pairing is shuffled, and the whole process is repeated. This helps to check for consistency of the data, to check for any false alerts made by the dog, to highlight those data inferences that might be due to dog/handler familiarity-or-unfamiliarity, handler cues, and to further help in the development of novel narcotic detector dog training programs. After each test session using different dogs and dog/handler combinations, both the simulants and the sniffing boxes in which they were confined are removed from the test site perimeter. The vacated spots are then replaced by new boxes, which are to be left unused for at least a 24-hour period. This ensures that the used grid-positions are aired for periods long enough to allow for scent-dissipation if grid contamination had occurred, and the position of scents were also changed after each trial period.

### Example 3

### Method of Making a Narcotic-Free Pseudoscent for Cocaine

After using GC/MS to determine the headspace scent composition of cocaine, the headspace scent composition was found to contain ethyl acetate and benzoic acid as two of the primary components. Thus, with the intention of forming a single-component pseudoscent for cocaine, a pseudoscent of cocaine was then formed by dispersing 2.40 g benzoic acid in 10 g of diatomaceous earth to produce a training aid with a headspace scent signature of benzoic acid within the 1 ft³ box. This pseudoscent was then validated using scent validity tests previously described, in order to ascertain its usability as a narcotic training aid. Using a search-and-detect technique, five out of five certified narcotics-detecting canines used in this study successfully detected this scent by showing behavioral cues normally associated with finding a narcotic material.

After the scent was validated, the simulant was subjected to canine narcotic-detection tests using 16 dogs that have been certified as narcotic-detector dogs. All canines used in this study successfully detected this scent by showing behavioral cues normally associated with finding a narcotic material, which was sitting down next to the box in a fashion they are taught to indicate when they liken a scent to that of a narcotic scent that they had been trained to detect. Note that none of the components used in this formulation is classified by DEA as a narcotic.

### Example 4

### Method of Making a Narcotic-Free Pseudoscent for Methamphetamine

Using GC/MS for headspace characterization, the headspace scent signature of Methamphetamine was found to contain benzaldehyde, benzylmethalamine and propiophenone as the primary components. Thus, with the intention of forming a two-component pseudoscent for Methamphetamine, a pseudoscent of Methamphetamine was then formed by dispersing 1.40 g propiophenone and 1.5 g benzaldehyde in 16 g of diatomaceous earth. This pseudoscent was then subjected to validation using scent validity tests previously described, in order to ascertain its usability as a narcotic training aid. Using a search-and-detect technique, six out of six certified canines used in this study successfully detected this scent by showing behavioral cues normally associated with finding a narcotic material.

After the scent validation process, the simulant was subjected to canine narcotic detection tests, using 16 dogs that have been certified as narcotic-detector dogs. Sixteen out of sixteen canines used in this study successfully detected this scent by showing behavioral cues normally associated with finding an narcotic material, which was sitting down next to the box in a fashion narcotic-detector dogs are taught to indicate when they liken a scent to that of an narcotic scent that they had been trained to detect. Note that none of the components used in this formulation is classified by DEA as a narcotic.

### Example 5

### Method of Making a Narcotic-Free Pseudoscent for Heroin

Using GC/MS for headspace characterization, the headspace scent signature of heroin was found to contain acetic acid and phenyl acetate as being among the primary components. A pseudoscent of Heroin was then formed by dispersing 4 g of phenyl acetate in 16 g of diatomaceous earth. This pseudoscent was then subjected to validation using scent validity tests previously described in order to ascertain its usability as a narcotic training aid. Using a search-and-detect technique, five out of five canines used in this study successfully detected this scent by showing behavioral cues normally associated with finding a narcotic material.

After the scent validation process, the simulant was subjected to canine narcotic detection tests using 20 dogs that have been certified as narcotic-detector dogs. All canines used in this study successfully detected this scent by showing behavioral cues normally associated with finding a narcotic material, which is sitting down next to the box in a fashion narcotic-detector dogs are taught to indicate when they liken a scent to that of a narcotic scent that they have been trained to detect. Note that none of the components used in this formulation is classified by DEA as a narcotic.

The examples and embodiments described herein are for illustrative purposes only and various modifications or changes in light thereof will be suggested to persons skilled in the art and are to be included within the spirit and purview of this application and scope of the appended claims. All publications, patents, and patent applications cited herein are hereby incorporated by reference for all purposes in their entirety.

1. A method for producing an non-narcotic pseudoscent of a narcotic, the method comprising:
a) Targeting a narcotic material for detection;
b) Identifying the non-narcotic components of the scent signature of the narcotic material that is targeted for detection;
c) Substituting the narcotic components within the scent signature of the narcotic material targeted for detection with non-narcotic components that are odoriferously similar to the narcotic components within such scent signature by replacing one or more of such narcotic components with a non-narcotic replacement that has similar electronic properties but are non-narcotic in character; and
d) Combining components identified in b) into a first formulation,
e) Combining the components identified in c) into a second formulation to form a non-narcotic pseudoscent wherein the non-narcotic pseudoscent is free of the narcotic.

2. The method of claim 1, further comprising combining the non-narcotic components of the scent signature of the first formulation with the second formulation to form a composite non-narcotic formulation.

3. The method of claim 1, wherein the non-narcotic replacement of the scent signature is selected from a narcotic component from the scent signature of the narcotic material.

4. The method of claim 3, wherein the narcotic component of the scent signature of the narcotic material is replaced with a non-narcotic equivalent successively by replacing the functional groups within the molecular structure of the narcotic component of the scent signature with functional groups that have similar electronic properties.

5. The method of claim 3, wherein the narcotic component of the scent signature of the narcotic material is replaced with a non-narcotic equivalent by replacing the molecular structural backbone of the narcotic component with a structural homologue that has similar electronic properties.

6. The method of claim 4, wherein electron withdrawing functional groups within the molecular structure of the narcotic component of the scent signature are replaced with functional groups with similar electron withdrawing properties

7. The method of claim 4, wherein electron-donating functional groups within the molecular structure of the narcotic component of the scent signature are replaced with functional groups with similar electron donating properties

8. The method of claim 6, wherein the electron-withdrawing replacement functional group is selected from the group consisting of cyano, acetyl, halogen, nitro, aldehyde, and carboxylic acid functional groups.

9. The method of claim 7, wherein the electron-donating replacement functional group is selected from the group consisting of amino, hydroxyl, alkoxy, halogen, aryl, and alkyl functional groups.

10. The method of claim 1, wherein the narcotic material is selected from the group consisting of cocaine, crack cocaine, K2 (spice), heroin, methamphetamines, amphetamines, methylenedioxymethamphetamines (MDMA), cannabis, ergoline, morphine, thebaine, synthetic cannabinoids, opium, lysergic acid diethylamide (LSD), hashish, and PCP.

11. The method of claim 1, wherein the non-narcotic components are selected from the group consisting of phenylacetic acid, cryophyllene, piperonal, cinnamic acid, benzodioxole, myrcene, propiophenone, butoxyethanol, terpineol, pyrrolidine, propyl benzoate, bis(hexamethylene)triamine, benzaldehyde, aminophenyl butane, benzylmethylamine, pinene, carene, limoline, aminophenylbutane, phenyl acetate, quinoline, hydroxyphenylacetic acid, methybenzylacetate, dipropylamine, diethylacetamide, acetylphenol, benzylideneacetone, isosafrole, ethyl acetate, hydroxyacetophenone, methyl cinnamate, propyl acetate, benzoic acid, phenylcyclohexene, methylenedioxyphenylacetic acid, water, and dimethylfornamide.

12. The method of claim 2, further comprising dissolving the first, second, or composite formulations in a solvent, adding an inert matrix, and removing the solvent.

13. The method of claim 2, wherein the second formulation is selected from the group consisting of phenylacetic acid, piperonal, isosafrole, benzodioxole, myrcene, pinene, cryophyllene, propiophenone, benzaldehyde, water, aminophenyl butane, N-Benzylmethylamine, aminophenylbutane, phenyl acetate, 2-hydroxyacetophenone, butoxyl ethanol, methyl cinnamate, propyl acetate, cinnamic acid, benzoic acid, 1-phenylcyclohexene, and dimethylfornamide.

14. The method of claim 1, further comprising compounding the first formulation, the second formulation, or the composite formulations with a chemically inert polymeric binder or dispersant with a molecular weight of ≧320 atomic mass units (a.m.u.).

15. The method of claim 1, further comprising absorbing the first formulation, the second formulation, or the composite formulations into a chemically inert porous supporting matrix material.

16. The method of 1, further comprising absorbing the first formulation, the second formulation, or the composite formulations onto grain husks, cellulose and natural fibers.

17. The method of claim 1, further comprising mixing the first formulation, the second formulation, or the composite formulation with a gelling agent to form a gel.

18. The method of claim 2, further comprising mixing the first formulation or the composite formulation to form an emulsion.

19. A method for producing a non-narcotic pseudoscent composition of a narcotic, the method comprising:
a. Identifying the non-narcotic components within the headspace scent signature of the narcotic material;
b. Selecting all or some of the identified non-narcotic components for combining into a formulation;
c. Combining non-narcotic components that are selected as odoriferously identical to the components of the headspace scent signature into a second formulation, wherein the non-narcotic pseudoscent is free of narcotics.

20. The composition of claim 19, wherein the non-narcotic components are selected from the group consisting of phenylacetic acid, piperonal, isosafrole, benzodioxole, myrcene, pinene, cryophyllene, propiophenone, benzaldehyde, aminophenyl butane, Benylmethylamine, aminophenyl butane, phenyl acetate, ethy benzoate, cyclohexylpiperidine, hydroxyacetophenone, butoxyl ethanol, water, methyl cinnamate, propyl acetate, cinnamic acid, benzoic acid, and phenylcyclohexene.

21. A method of evaluating a non-narcotic pseudoscent of a narcotic by exposing an effective amount of a non-narcotic pseuodoscent to a creature that is certified in a narcotics detection program that uses real narcotics in its training process.

22. The method of claim 22, wherein the creature is selected from the group consisting a bee, dog, and insect.
